(19) United States
(12) Patent Application Publication
(10) Pub. No.: US 2010/0151558 A1
(43) Pub. Date: Jun. 17, 2010

(54) Tubular Microbial Growth System

(75) Inventors: Gary A. Alianell, Villa Park, CA (US); Frederic F. Derwitsch, Melbourne, FL (US); Everett E. Howard, Fellsmere, FL (US)

Correspondence Address: DAVIS WRIGHT TREMAINE LLP - San Francisco, 505 MONTGOMERY STREET, SUITE 800, SAN FRANCISCO, CA, 94111, US

(73) Assignee: PETROALGAE, LLC, Melbourne, FL (US)

(21) Appl. No.: 12/441,115

(22) PCT Filed: Sep. 13, 2007
(86) PCT No.: PCT/US07/20211

§ 371 Date: Dec. 10, 2009

Related U.S. Application Data
(60) Provisional application No. 60/825,475, filed on Sep. 13, 2006.

(51) Int. Cl. C12N 1/12; C12M 1/00

(52) U.S. Cl. 435/257.3; 435/292.1; 435/257.1

(57) ABSTRACT

Systems and methods for microorganism growth are disclosed. The systems include continuous-culture processes for the growth of large volumes of microorganisms.

### RELATED APPLICATIONS

This application claims priority from U.S. Provisional Application 60/825,475, filed Sep. 13, 2006, incorporated by reference herein in its entirety.

### FIELD OF THE INVENTION

The present invention generally relates to systems and methods for growing microorganisms such as algae and cyanobacteria, and in particular to a system and method of growing microorganisms using a tubular or pipe-like bioreactor pipe system that is inoculated with a starter culture from a bioreactor.

### BACKGROUND OF THE INVENTION

Current microorganism growth methods typically include photo-bioreactors which can achieve high yield but have an associated high capital cost. Alternative growth methods include natural ponds, which have the advantage of low capital cost, but also the disadvantage of low yield. Embodiments of the invention provide a hybrid growth system and method which can achieve high yields at lower costs than current systems.

### SUMMARY OF THE INVENTION

Embodiments of the invention include system for growing microorganisms that includes a tubular vessel made of a substantially clear flexible, material; an inlet for adding media; an outlet for harvesting the microorganisms; an energy source; a media supply; and a microorganism that can be selected from the following: Pseudochlorococcum sp., Chlorococcum sp., Chlorella sp., Scenedesmus sp., Palmellococcus sp., Cylindrospermopsis sp., Planktothrix sp. And the like. In various embodiments of the invention, the substantially clear, flexible material can be polyethylene. In various embodiments of the invention, the substantially clear, flexible material can be PEEK. In various embodiments of the invention, the substantially clear, flexible material can be an ultraviolet-resistant material.

In various embodiments of the invention, the substantially clear, flexible material can be coated to selectively pass specific wavelengths of light, or coated to selectively pass green light and reflect blue light. In various embodiments of the invention, the substantially clear, flexible material can be coated to selectively pass visible light of a wavelength of about 510 nm and reflect visible light of a wavelength of about 475 nm. In some embodiments of the invention, the substantially clear, flexible material can be coated to passes blue light and reflect green light. In various embodiments of the invention, the substantially clear, flexible material can be coated to selectively pass visible light of a wavelength of about 475 nm and reflect visible light of a wavelength of about 510 nm. In some embodiments the objective of the coating is to reduce the amount of heat-generating light, while in other embodiments the objective is to increase the amount of heat-generating light.

In various embodiments of the invention, the energy source can include combustion of the biomass produced by the system, or include ethanol produced from the biomass of the system.

In various embodiments of the invention, the media can be waste-water, including CAFO waste-water. In various embodiments of the invention, the microorganism can be Pseudochlorococcum sp. In various embodiments of the invention, the microorganism can be Chlorella sp.

Various embodiments of the invention include a method for growing microorganisms that can include adding media to a substantially clear, flexible, tubular vessel, and sterilely inoculating the tubular vessel with a microorganism that can be selected from the group including Pseudochlorococcum sp., Chlorococcum sp., Chlorella sp., Scenedesmus sp., Palmellococcus sp., Cylindrospermopsis sp., and Planktothrix sp. Various embodiments of the invention can include means for monitoring a parameter of the culture such as pH, temperature, O₂ concentration, CO₂ concentration, NO₃⁻/PO₄³⁻ levels, conductivity, or turbidity. Some embodiments of the invention can include harvesting at least a part of the culture when the culture exceeds a parameter such as pH, temperature, O₂ concentration, CO₂ concentration, NO₃⁻/PO₄³⁻ levels, conductivity, or turbidity.

In some embodiments of the invention, the tubular vessel material can be substantially clear, and/or flexible. In various embodiments of the invention, the substantially clear, flexible material can be coated to selectively pass specific wavelengths of light, or coated to selectively pass green light and reflect blue light. In various embodiments of the invention, the substantially clear, flexible material can be coated to selectively pass visible light of a wavelength of about 510 nm and reflect visible light of a wavelength of about 475 nm. In some embodiments of the invention, the substantially clear, flexible material can be coated to passes blue light and reflect green light. In various embodiments of the invention, the substantially clear, flexible material can be coated to selectively pass visible light of a wavelength of about 475 nm and reflect visible light of a wavelength of about 510 nm. In various embodiments of the invention, the substantially clear, flexible material can be polyethylene. In various embodiments of the invention, the substantially clear, flexible material can be PEEK. In various embodiments of the invention, the substantially clear, flexible material can be an ultraviolet-resistant material.

In various embodiments of the invention, the energy source can include combustion of the biomass produced by the system, or include ethanol produced from the biomass of the system.

In various embodiments of the invention, the media can be waste-water, including CAFO waste-water. In various embodiments of the invention, the microorganism can be Pseudochlorococcum sp. In various embodiments of the invention, the microorganism can be Chlorella sp.

Some embodiments of the invention can include an apparatus for growing microorganisms, and such apparatus can include a substantially clear, flexible, tubular vessel. In various embodiments of the invention, the substantially clear, flexible vessel can be made of polyethylene. In various embodiments of the invention, the substantially clear, flexible vessel can be made of PEEK. In various embodiments of the invention, the substantially clear, flexible material can be an ultraviolet-resistant material.

In some embodiments of the invention, the apparatus can include means for introducing media to the apparatus, and/or means for introducing an microorganism such as Pseudochlorococcum sp., Chlorococcum sp., Chlorella sp., Scenedesmus sp., Palmellococcus sp., Cylindrospermopsis sp., or Planktothrix sp. to the culture. Various embodiments of the invention can include means for monitoring parameters of the culture, and such parameters can include pH, temperature, O₂ concentration, CO₂ concentration, NO₃⁻/PO₄³⁻ levels, conductivity, and turbidity. Various embodiments of the invention can include means for harvesting at least a part of the culture when the culture exceeds a parameter such as pH, temperature, O₂ concentration, CO₂ concentration, NO₃⁻/PO₄³⁻ levels, conductivity, or turbidity.

In some embodiments of the invention, the bioreactor pipe material can be substantially clear, and/or flexible. In various embodiments of the invention, the substantially clear, flexible material can be coated to selectively pass specific wavelengths of light, or coated to selectively pass green light and reflect blue light. In various embodiments of the invention, the substantially clear, flexible material can be coated to selectively pass visible light of a wavelength of about 510 nm and reflect visible light of a wavelength of about 475 nm. In some embodiments of the invention, the substantially clear, flexible material can be coated to passes blue light and reflect green light. In various embodiments of the invention, the substantially clear, flexible material can be coated to selectively pass visible light of a wavelength of about 475 nm and reflect visible light of a wavelength of about 510 nm. In various embodiments of the invention, the substantially clear, flexible material can be polyethylene. In various embodiments of the invention, the substantially clear, flexible material can be PEEK. In various embodiments of the invention, the substantially clear, flexible material can be an ultraviolet-resistant material.

In various embodiments of the invention, the energy source can include combustion of the biomass produced by the system, or include ethanol produced from the biomass of the system.

In various embodiments of the invention, the media can be waste-water, including CAFO waste-water. In various embodiments of the invention, the microorganism can be Pseudochlorococcum sp. In various embodiments of the invention, the microorganism can be Chlorella sp.

Various embodiments of the invention can include multiple systems for growing microorganisms.

### BRIEF DESCRIPTION OF THE DRAWINGS

FIG. 1 is a partial diagrammatical illustration of a bioreactor pipe portion a tubular microbial growth system in keeping with the teachings of e present invention.

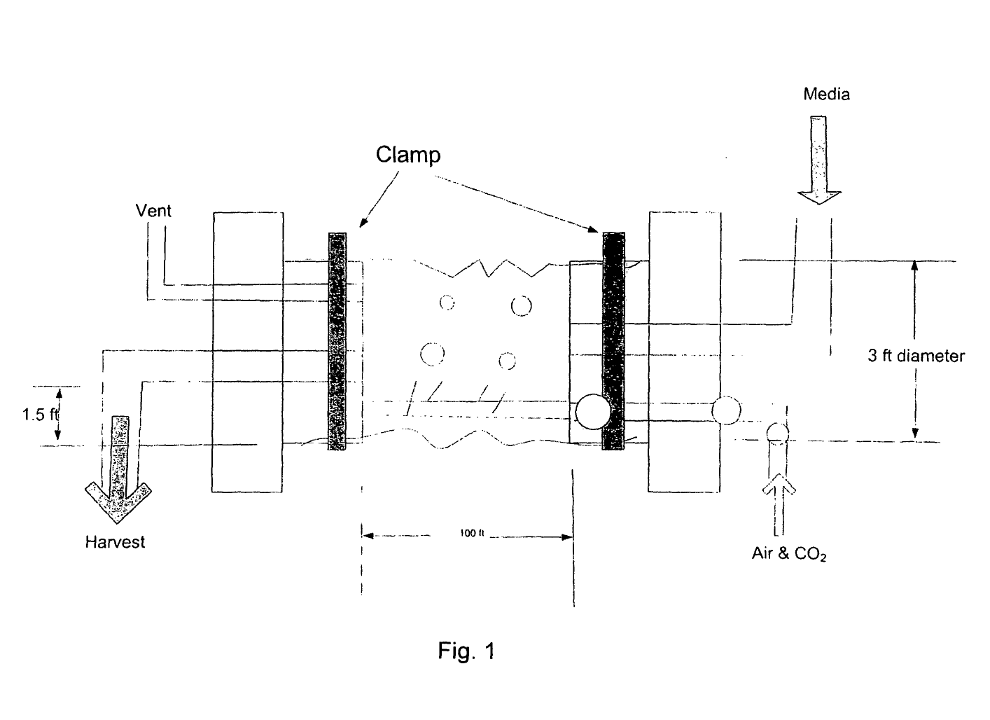

FIG. 2 is a diagrammatical perspective view of a portion of the bioreactor pipe in FIG. 1.

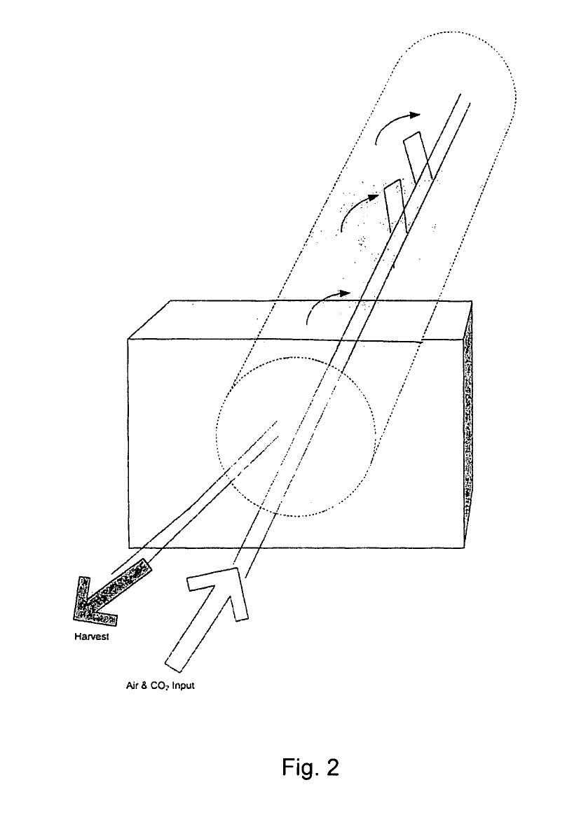

### DESCRIPTION OF EXEMPLARY EMBODIMENTS OF THE INVENTION

The present invention relates a method for continuous harvest of microorganisms on a large scale. Numerous tubular growth systems can each be seeded from a single sterile “nursery” bioreactor, and their growth cycles can be offset between each tubular growth unit (bioreactor pipe) such that there is always at least one bioreactor pipe ready for harvest each day. Other harvesting methods are also contemplated, for example, a bioreactor pipe can be continuously harvested by withdrawing culture at a set rate, while continuously adding a similar volume of media. For purposes of simplicity, the term “microorganism” shall be used in this application; however, it should be understood that the term “microorganism” can mean, for example, algae, cyanobacteria, or the like.

Examples of algal growth systems are disclosed in Provisional U.S. Patent Applications Nos. 60/782,564, filed on Mar. 15, 2006; 60/825,464, filed on Sep. 13, 2006; and 60/825,592, filed on Sep. 14, 2006; U.S. patent application Ser. No. 11/728,297, filed on Mar. 15, 2007; and PCT Application No. PCT/US2007/006466, filed on Mar. 15, 2007, both entitled “SYSTEMS AND METHODS FOR LARGE-SCALE PRODUCTION AND HARVESTING OF OIL-RICH ALGAE,” each of which is hereby incorporated by reference in its entirety.

Embodiments of the invention are directed to systems and methods for growing microorganisms. Tubular growth systems including bioreactor pipes can provide a low-cost, high-efficiency means for producing biodiesel. The bioreactor pipes are seeded with a volume of inoculum from a sterile photo-bioreactor via a closeable pipe, creating a microorganism culture. The microorganisms are allowed to grow for the preferred time to reach an optimum phase. At a time point where optimal yield of target (in terms of biomass or byproduct) has been achieved, the microorganisms can be harvested.

Gravitational flow can provide movement of the culture to the harvesting collection area. A set volume of culture can be moved to a harvest tank fitted with a filter. As the culture passes over the filter, the target microorganisms can be collected. Depending on the filter, the target can be found on the filter or in the liquid that passes. If the latter, a centrifugation step can be included to separate the target microorganisms from the liquid. Other methods of microorganism collection are contemplated. For example, flocculation can also be used to collect the microorganisms. These chemicals cause algae in liquids to aggregate, forming a floc, and thus increasing the sedimentation of the suspended algae.

Once the liquid media has been separated, the microorganisms or target products can be mechanically (by, for example, physical grinding, pressing in a French press machine or equivalent structure, or the like), chemically, or by sonication, processed to extract desired products including oils. Examples of extraction of products include, for example, the use of supercritical carbon dioxide or propane (ref: U.S. Pat. No. 5,539,133, G. Kohn, et al., Jul. 23, 1996.), hexane or ethanol solvent, mechanical expression, ultrasonic waves, chemical lysis, and the like.

By way of example of a commercially viable product, there is increasing interest in bio-diesel as an alternative to petro-diesel. There are two problems with this approach: first, this would displace the food crops grown to feed mankind and second, traditional oilseed crops are neither the most productive nor the most efficient source of vegetable oil. However, microalgae is, by a factor of 8 to 25 for palm oil and a factor of 40 to 120 for rapeseed, the highest potential energy yield temperate vegetable oil crop. Micro-algae are the fastest growing photosynthesizing organisms, and can complete an entire growing cycle every few days. Further, algae does not compete with agriculture for nutrients, requiring neither farmland nor fresh water.

Algae contain fat, carbohydrates, and protein. Some contain up to 60% fat, and under some conditions as much as 70% of that amount can be recovered. In other conditions, more than 70% of the fat present in the algal cell can be recovered. After the fat is harvested, the oil can be used as a source of, for example, fatty acids, detergent applications, bio-diesel, palm and soy oil alternatives, and the like. Under stress conditions, some algae can produce high grade pigments. These pigments can be isolated during the harvest or processing step and used in areas such as, for example, pharmaceutical encapsulation, medical imaging, food coloring, and the like. The algal bodies can be used as fertilizer, in food products, or directly burned to generate electricity. In some embodiments, the algal bodies can be used to produce cellulosic ethanol.

In some embodiments of the invention, the microorganism is an alga. Algae are a diverse group of eukaryotic organisms that contain chlorophyll and carry out photosynthesis. Some contain other photosynthetic pigments which can give the organisms a characteristic color. Algae occur in a wide range of forms from microscopic to macroscopic e.g. seaweeds, some of which are up to 30 meters long. Microscopic algae exist as, for example, single cells e.g. diatoms, in colonies e.g. Volvox or in filaments e.g. Spirogyra, and the like. Embodiments of the invention utilize algae that can grow photosynthetically utilizing CO₂ and sunlight, in addition to a minimum amount of trace nutrients. In some embodiments of the invention, the microorganism is a cyanobacterium. Cyanobacteria are prokaryotic organisms and include unicellular as well as colonial species. Embodiments of the invention utilize cyanobacteria that can grow photosynthetically utilizing CO₂ and sunlight, in addition to a minimum amount of trace nutrients.

Various embodiments of the invention can utilize, for example, microorganism strains such as, for example, Pseudochlorococcum sp., Chlorococcum sp., Chlorella sp., Scenedesmus sp., Palmellococcus sp., Cylindrospermopsis sp., and Planktothrix sp., and the like.

Some embodiments of the invention include a bioreactor pipe designed for growth of microorganisms. Suitable bioreactor pipes can be of various dimensions, provided they allow sufficient sunlight to penetrate the inside of the bioreactor pipe and provide enough interior volume such that a circulation current can be created. For example, the bioreactor pipe can be between 25′ and 300′ in length, between 50′ and 200′ in length, or between 75′ and 150′ in length. In some embodiments of the invention, the bioreactor pipe can be 100′ in length. In some configurations of bioreactor pipe length, diameter, and/or thickness, pressures can vary throughout the bioreactor pipe, however, pressure variations be addressed with changes in bioreactor pipe parameters, such as, for example, diameter, bioreactor pipe slope, and the like.

The bioreactor pipe can be made from any suitable materials, including, for example, polyethylene, polyetheretherketon (PEEK), clear elastomers, or the like. The material can be of a thickness sufficient to withstand the abrasive effects of, for example, agitation forces, aeration forces, external handling forces, and the like. The thickness of the material can be, for example, between 4 mils and 15 mils, or between 6 mils and 12 mils, or between 8 mils and 10 mils. In some embodiments of the invention, the thickness of the material can be 8 mils. In some embodiments, the bioreactor pipe can be disposable. In some embodiments, the bioreactor pipe can be ultraviolet (UV)-treated to resist the damaging effects of sunlight.

In some embodiments of the invention, the location for placing the bioreactor pipe can be a flat area, with raised berms at either end. The distance between the raised berms can be between 25′ and 300′, between 50′ and 200′, or between 75′ and 150′. In some embodiments, the distance between the berms can be 100′. In some embodiments the location can be completely flat. In other embodiments, the location can be a depression in the ground, for example a draw, valley, culvert, or the like. The long axis of the bioreactor pipe can be oriented in a specific direction, depending upon the time of year and the latitude of the location area. For example, the long axis of the bioreactor pipe can be laid, for example, North-to-South, East-to-West, Northeast-to-Southwest, or the like.

In some embodiments of the invention, a shallow trough is prepared along the desired axis of the bioreactor pipe. The trough can limit the lateral movement of the bioreactor pipe. In some embodiments, lateral movement of the bioreactor pipe can be limited by, for example, wedges, frames, stakes, or the like. The trough can be prepared by, for example, hand digging, a backhoe, bulldozing, dragging a prepared form, or the like. Multiple troughs can be prepared, to accommodate multiple bioreactor pipes, and the troughs can be spaced apart to eliminate or minimize shadowing upon the bioreactor pipe surfaces. The ends of the bioreactor pipe can be placed such that they are higher than the rest of the bioreactor pipe. For example, the bioreactor pipe end can be placed upon a berm. The bioreactor pipe can be set at a slight incline, for example 3 inches for every 100′, to make use of gravity for drainage purposes.

The distance between the sides of a bioreactor pipe is the “light path,” which affects sustainable microorganism concentration, photosynthetic efficiency, and biomass productivity. In various embodiments, the light path of a bioreactor pipe can be between 6″ and 42″, or between 12″ and 36″, or between 18″ and 30″, or between 22″ and 26″ in diameter. In some embodiments of the invention the light path is 24″. The optimal light path for a given application will depend, at least in part, on factors including the specific microorganism strains to be grown and/or specific desired products to be produced.

Some embodiments of the invention can include at least one external power source for operating, for example, pumps, sensors, control units, and the like. Suitable power sources can include, for example, solar power, hydroelectric power, wind power, battery power, combustion-based power, utility-provided power, and the like. In some embodiments, biomass produced by the bioreactor is processed then burned to provide at least a portion of the electrical power used by the system. In some embodiments of the invention, carbon credits can be accrued through use of the system.

Embodiments of the invention can include a water supply. The water supply can be recycled from prior uses. The water supply can be treated before use in the system, and such treatments can include, for example, ultraviolet radiation, ozone, ultrasound, filtration, hollow fiber filtration, sand filtration, gravel filtration, diatomaceous earth, activated charcoal, and the like. The water supply can include various nutrients.

In certain embodiments of the invention, nutrients are added to the water supply prior to adding the water to the bioreactor pipe. In certain embodiments, added nutrients can include, for example, carbon, nitrates, phosphates manganese, magnesium, potassium, phosphorous, and the like. In some embodiments of the invention, the system includes a feedback loop that measures the level of selected nutrients, for example, carbon, nitrates, phosphates manganese, magnesium, potassium, phosphorous, and the like, and adds nutrients if threshold levels are not met.

The appropriate volume of growth medium can be any volume suitable for cultivation of the microorganism for any purpose, whether for standard laboratory cultivation, or large scale cultivation for use in, for example, bioremediation, lipid production, algal biomass production, or the like. Suitable microorganism growth medium can be any such medium, including, for example, BG-11 growth medium, and the like.

Examples of suitable media include, but are not limited to, Luria Broth, brackish water, water having nutrients added, dairy runoff, media with salinity of less than or equal to 1%, media with salinity of greater than 1%, media with salinity of greater than 2%, media with salinity of greater than 3%, media with salinity of greater than 4%, and combinations thereof. Nitrogen sources can include, for example, nitrates, ammonia, urea, nitrites, ammonium salts, ammonium hydroxide, ammonium nitrate, monosodium glutamate, soluble proteins, insoluble proteins, hydrolyzed proteins, animal byproducts, dairy waste, casein, whey, hydrolyzed casein, hydrolyzed whey, soybean products, hydrolyzed soybean products, yeast, hydrolyzed yeast, corn steep liquor, corn steep water, corn steep solids, distillers grains, yeast extract, oxides of nitrogen, nitrous oxide, and the like. Carbon sources can include, for example, sugars, monosaccharides, disaccharides, sugar alcohols, fats, fatty acids, phospholipids, fatty alcohols, esters, oligosaccharides, polysaccharides, mixed saccharides, glycerol, carbon dioxide, carbon monoxide, starch, hydrolyzed starch, and the like.

Additional media ingredients can include buffers, minerals, growth factors, anti-foam, acids, bases, antibiotics, surfactants, materials to inhibit growth of undesirable cells, and the like. In certain embodiments, no nutrients are added to the water supply.

In various embodiments of the invention, the growth medium useful for culturing the microorganism comprises waste-water or waste gases. In some embodiments, when waste-water is used to prepare the medium, the waste-water is nutrient-contaminated (e.g., industrial waste-water, agricultural waste-water domestic waste-water, contaminated groundwater and surface water). In some embodiments, the growth medium includes waste gases emitted from power generators burning natural gas or biogas, or flue gas emissions from fossil-fuel-fired power plants. In some embodiments, the microorganism can be first cultivated in a primary growth medium, followed by addition of waste-water and/or waste gas. Alternatively, the microorganism. can be cultivated solely in the waste stream source. When a particular nutrient or element is added into the culture medium, it will be taken up and assimilated by the microorganism just like other nutrients. Ultimately, both waste-water-contained and -added nutrients are removed and converted into macromolecules (such as lipids, proteins, or carbohydrates) stored in microorganism biomass.

In some embodiments, waste-water is added to the culture medium at a desired rate. This water, being supplied from the waste-water source, can contain additional nutrients, such as phosphates, and/or trace elements (such as iron, zinc), which supplement growth of the microorganism. In one embodiment, if the waste-water being treated contains sufficient nutrients to sustain the microorganism growth, it can be possible to use less of the growth medium. As the waste-water becomes cleaner due to microorganism uptake of nutrients, the amount of growth medium can be increased. Factors affecting waste-water input rate include microorganism growth rate, light intensity, culture temperature, initial waste-water nutrient concentrations; and the specific algal uptake rate of certain nutrient(s).

In other embodiments of the invention, waste-water can come from Concentrated Animal Feeding Operations (CAFOs), such as dairy farms, which can contain high concentrations of ammonia (hundreds to thousands of milligrams per liter of nitrogen as ammonia) and phosphate (tens to hundreds of milligrams per liter of phosphorous as phosphate). Full-strength CAFO waste-water can be used as a “balanced growth medium” for sustaining rapid growth of selected microorganism strains in bioreactor pipes as described above. In some cases the CAFO waste-water can be diluted to a certain extent to accelerate growth and proliferation of the microorganism of the present invention.

In some embodiments of the invention, the carbon source can be CO₂ derived from, for example, fermentation, reduction of calcium carbonate, sublimation of dry ice, mines, or the like. In some embodiments of the invention, CO₂ can be provided by way of micro-bubbling or aeration. In some embodiments, CO₂ is added to the system from cylinders.

The pH of the culture can be controlled through the use of a buffer, or by addition of an acid or base at the beginning or during the course of the growth cycle. In some cases, both an acid and a base can be used in different zones of the bioreactor pipe or in the same zone at the same or different times in order to achieve a desirable degree of control over the pH. Non-limiting examples of buffer systems include, for example, phosphate, TRIS, TAPS, bicine, tricine, HEPES, TES, MOPS, PIPES, cacodylate, MES, acetate, and the like. Non-limiting examples of acids include, for example, sulfuric acid, hydrochloric acid, lactic acid, acetic acid, and the like. Non-limiting examples of bases include, for example, potassium hydroxide, sodium hydroxide, ammonium hydroxide, ammonia, sodium bicarbonate, calcium hydroxide, sodium carbonate, and the like. Some of these acids and bases, in addition to modifying the pH, can also serve as nutrients for the cells. The pH of the culture can be controlled to approximate a constant value throughout the entire course of the growth cycle, or it can be changed during the growth cycle. Such changes can be used, for example, to initiate or terminate different molecular pathways, to force production of one particular product, to force accumulation of a product such as fats, dyes, or bioactive compounds, to suppress growth of other microorganisms, to suppress or encourage foam production, to force the cells into dormancy, to revive them from dormancy, or the like.

In some embodiments of the invention, the pH of the culture can be between 4.0 and 10.0, or between 5.0 and 8.0, or between 6.0 and 7.0. In some embodiments of the invention, the pH can be 6.5.

Likewise, the temperature of the culture can in some embodiments be controlled to approximate a particular value, or it can be changed during the course of the fermentation for the same or different purposes as listed for pH changes. In certain of such embodiments, a temperature control device can be provided that comprises a temperature measurement component that measures a temperature within the system, such as a temperature of the medium, and a control component that can control the temperature in response to the measurement. The control component can comprise an internal submerged coil or an external jacket on the side or bottom of the bioreactor pipe. In some embodiments of the invention,

In some embodiments of the invention, culturing temperatures of between 10° and 40° C. are used; in other embodiments, temperature ranges between 15° and 30° are used, and in other embodiments, temperature ranges between 20° and 25° are used. In some embodiments, the culturing temperature can be 25° C.

Similarly, in certain embodiments, a light intensity between 20 μmol m⁻²s⁻¹ to 1000 μmol m⁻²s⁻¹ is used; in various other embodiments, the range can be 100 μmol m⁻²s⁻¹ to 500 μmol m⁻²s⁻¹ or 150 μmol m⁻²s⁻¹ to 250 μmol m⁻²s⁻¹. Further, in some embodiments of the invention, aeration is carried out with between 0% and 40% CO₂; in various other embodiments, aeration is carried out with between 0.5% and 10% CO₂, 0.5% to 5% CO₂, or 0.5% and 2% CO₂.

Certain embodiments of the system can contain a mechanism for agitating the microorganisms. In some embodiments of the invention, a pump is used to force media through the bioreactor pipe. Suitable pumps can include, for example, peristaltic pumps, lift pumps, and the like. Turbulent flow can be created through use of, for example, a diameter change inside the bioreactor pipe, or the like. In certain embodiments, agitation can be caused by pumping the media through a smaller pipe within the larger bioreactor pipe. In certain embodiments, this smaller pipe can have openings oriented toward the center of the bioreactor pipe. In certain embodiments, openings in the center of the smaller pipe can force pumped media to flow along the sides of the bioreactor pipe and intersect at or near the top of the bioreactor pipe. In some embodiments, angled baffles can extend from the sides of the bioreactor pipe toward the center, and can create turbulent flow patterns within the bioreactor pipe.

Embodiments of the invention can contain a mechanism for aerating the microorganisms. The use of the term “aeration” within this description is meant to encompass all forms of delivery of a gas to the cells of the culture in the bioreactor pipe. The gas being delivered can include, for example, air, oxygen, carbon dioxide, carbon monoxide, oxides of nitrogen, nitrogen, hydrogen, inert gases, exhaust gases such as from power plants, and the like. The gas can be pressurized or not, and can be bubbled or sparged, introduced to the surface of the fermentation culture, created in situ, or diffused through a porous or semi-permeable membrane or barrier. In some embodiments, a smaller pipe within the larger bioreactor pipe can carry gas within the larger bioreactor pipe. In some embodiments, from this smaller pipe protrude hollow, flexible hoses with weighted ends. These hoses will swing back-and-forth in a random manner when pressurized gas is forced through them. In certain embodiments, the incoming gas can be heated or cooled to help maintain appropriate growth conditions for the microorganism. In some embodiments, cooling of the gas can be achieved by burying the gas line to a depth sufficient that groundwater covers the line. In some embodiments, a water trough can be used to cool the gas line. In some embodiments, heating the gas line can be achieved by, for example, exposure to sunlight, exposure to heated water, or the like.

In some embodiments of the invention, the mechanism for mixing, aeration and/or current flow can be, for example, baffles, mixing foils, air lifts, slotted vented pipes, or the like. The injection of the air results in a mixture of air bubbles and water, which being lighter in weight than water outside the discharge pipe, forces the air/water mixture up. In some embodiments of the invention CO₂ will be injected into the air stream to as it can be necessary for growth and reproduction of the algae.

In some embodiments of the invention, the bioreactor pipe can be coated with, for example, paint, pigment, plastic, or the like. In some embodiments, the bioreactor pipe coating can be designed to pass certain wavelengths of light while reflecting other wavelengths. For example, the bioreactor pipe can be coated in such a way as to reflect light of a certain color while passing light of other colors through. In some embodiments, the ends of the bioreactor pipe can be designed to allow for immobilization such that the bioreactor pipe rests above or on top of the ground. In some embodiments the bioreactor pipe can include a pressure release mechanism.

In some embodiments of the invention the ends of the bioreactor pipe can be sealed with, for example, rigid caps, cable ties, or the like. In certain embodiments, the bioreactor pipe ends can include ports through which data and power lines my be placed. The data and power lines can include, for example, ethernet cable, optical cable, coaxial cable, and the like. In some embodiments, data can be transmitted from within the bioreactor pipe wirelessly.

In some embodiments of the invention, the bioreactor pipe includes an inlet port. This port can be used to add various materials to the reactor pipe, for example, culture medium, algal suspensions, water, waste-water, nutrient solutions, acids, bases, buffers, and the like can be added in this manner.

Some embodiments of the invention include an outlet port for removing materials from the bioreactor pipe, for example during harvesting, draining, cleaning, or the like.

In some embodiments of the invention, the bioreactor pipe includes sensors, such as, for example, pH, temperature, O₂ concentration, CO₂ concentration, NO₃⁻/PO₄³⁻ levels, conductivity, turbidity, or the like. In some embodiments, these sensors can transmit data outside the bioreactor pipe through means described above.

Certain embodiments of the invention include a control unit such as, for example, a computer, a terminal attached to a network, or the like. The control unit can record, track, and visually depict culture parameters such as, for example, pH, temperature, O₂ concentration, CO₂ concentration, NO₃⁻/PO₄³⁻ levels, conductivity, turbidity, or the like.

In some embodiments of the invention, the bioreactor pipe is supported by machinery capable of tilting the bioreactor pipe through both vertical and horizontal axes.

In some embodiments of the invention, the system includes a nursery bioreactor for inoculating the bioreactor pipe with microorganisms. The nursery bioreactor can be fluidly connected to the bioreactor pipe. In some embodiments the nursery bioreactor can be sterilely-operated.

In some embodiments of the invention, once the culture has achieved a sufficient degree of growth, the algae can be harvested. Harvest can occur directly from the bioreactor tube or after transfer of the culture to a storage tank. The harvesting steps can include, for example, killing the cells or forcing them into dormancy, separating the cells from the bulk of the media, drying the cells, lysing the cells, separating the desirable components, isolating the desired product, and the like. In some embodiments, not all of these steps are practiced together; various embodiments can combine various different steps and can also include additional steps and/or combinations of various functions into one or several steps. Additionally the steps actually practiced can be practiced in a different order than presented in this list.

Some embodiments of the invention employ a method for harvesting algae which utilizes commercially available equipment such as fixed media filters to remove loosely-adsorbed water to less than 50% weight. Then the retentate (retained by the filter medium) is compressed in a filter press to squeeze out the oil. By way of example, embodiments of the invention can include a bioreactor pipe having a suitable volume for producing a given number of l/day in a 50% bioreactor pipe volume.

In certain embodiments, killing or forced dormancy of the cells can be accomplished by a number of means depending on the cells and the product desired. Suitable means include, for example, heating, cooling, the addition of chemical agents such as acid, base, sodium hypochlorite, enzymes, sodium azide, antibiotics, or the like.

In some embodiments of the invention, separation of the cell mass from the bulk of the growth medium can be accomplished in a number of ways. Non-limiting examples include, screening, centrifugation, rotary vacuum filtration, pressure filtration, hydrocycloning, flotation, skimming, sieving, gravity settling, and the like. Other techniques, such as addition of precipitating agents, flocculating agents, or coagulating agents, can also be used in conjunction with these techniques. Flocculating agents can include, for example, iron, phosphatic clay, and the like. In some embodiments, the flocculating agent can be removed with, for example, a hydrocyclone, or the like, and then re-used. In some embodiments, the desired product will be in one of the streams from a separating device and in other cases it will be in the other stream. In some embodiments, two or more stages of separation can be performed. When multiple stages are used, they can be based on the same or a different technique. Non-limiting examples include screening of the bulk of the bioreactor pipe contents, followed by filtration or centrifugation of the effluent from the first stage.

In some embodiments of the invention, cell lysis can be achieved mechanically or chemically. Non-limiting examples of mechanical methods of lysis include pressure drop devices such as a French press or a pressure drop homogenizer, colloid mills, bead or ball mills, high shear mixers, thermal shock, heat treatment, osmotic shock, sonication, expression, pressing, grinding, expeller pressing and steam explosion. Non-limiting examples of chemical means include the use of enzymes, oxidizing agents, solvents, surfactants, and chelating agents. Depending on the exact nature of the technique being used, the lysis can be done dry, or a solvent such as, for example, water, or the like, or steam can be present. Solvents that can be used for the lysis or to assist in the lysis include, but are not limited to hexane, heptane, supercritical fluids, chlorinated solvents, alcohols, acetone, ethanol, methanol, isopropanol, aldehydes, ketones, chlorinated solvents, fluorinated-chlorinated solvents, and combinations of these. Exemplary surfactants include, but are not limited to, detergents, fatty acids, partial glycerides, phospholipids, lysophospholipids, alcohols, aldehydes, polysorbate compounds, and combinations of these. Exemplary supercritical fluids include, for example, carbon dioxide, ethane, ethylene, propane, propylene, trifluoromethane, chlorotrifluoromethane, ammonia, water, cyclohexane, n-pentane, toluene, and the like. The supercritical fluid solvents can also be modified by the inclusion of water or some other compound to modify the solvent properties of the fluid. Suitable enzymes for chemical lysis include proteases, cellulases, lipases, phospholipases, lysozyme, polysaccharases, and combinations thereof. Suitable chelating agents include, for example, EDTA, porphine, DTPA, NTA, HEDTA, PDTA, EDDHA, glucoheptonate, phosphate ions (variously protonated and non-protonated), and the like. In some cases, combinations of chemical and mechanical methods can be used.

In certain embodiments of the invention, separation of the lysed cells from the product-containing portion or phase can be accomplished by various techniques, for example, centrifugation, hydrocycloning, filtration, flotation, gravity settling, and the like. In some embodiments, it can be desirable to include a solvent or supercritical fluid, for example, to solubilize the desired product, reduce interaction between the product and the broken cells, reduce the amount of product remaining with the broken cells after separation, or to provide a washing step to further reduce losses. Suitable solvents include, for example, hexane, heptane, supercritical fluids, chlorinated solvents, alcohols, acetone, ethanol, methanol, isopropanol, aldehydes, ketones, and fluorinated-chlorinated solvents. Exemplary supercritical fluids include carbon dioxide, ethane, ethylene, propane, propylene, trifluoromethane, chlorotrifluoromethane, ammonia, water, cyclohexane, n-pentane, toluene, and the like, as well as combinations of these. The supercritical fluid solvents can also be modified by the inclusion of water or an other compound to modify the solvent properties of the fluid.

In some embodiments of the invention, it will be desirable to dry the cellular material prior to further processing. For example, drying can be desired when the subsequent processing occurs in a remote location or requires larger volumes of material than are provided by a single fermentation batch, or if the material must be campaigned through to achieve more cost-effective processing, or if the presence of water will cause processing difficulties such as emulsion formation, or for other reasons not listed here. Suitable drying systems include, for example, air drying, solar drying, drum drying, spray drying, fluidized bed drying, tray drying, rotary drying, indirect drying, direct drying, and the like.

In certain embodiments of the invention, methods used to clean, sanitize, and sterilize the bioreactor pipe include, for example, low-pressure steam, detergents, surfactants, chlorine, bleach, ozone, UV light, peroxide, and the like, and combinations thereof. In one embodiment, the bioreactor pipe can be rinsed with water, washed with a detergent, rinsed with water, sprayed with a bleach solution (sodium hypochlorite), and then filled with media and inoculum. In other embodiments, the bioreactor pipe can be filled with bleach solution and drained, and then the bleach solution can be neutralized with a reducing agent such as sodium thiosulfate.

In some embodiments, the invention can include a large-scale controlled continuous cultivation system with a sterile inoculation center (a photo-bioreactor by way of example) connected to a system of bioreactor pipes. The bioreactor pipes can have the bioreactor built in as a closed system. Each bioreactor pipe has connector seals on each end. A first end can provide media input and inoculum to the bioreactor pipe and a second end can include means to empty the bioreactor pipe of algae, thus harvesting the algae. Within one embodiment including a bioreactor pipe can be another pipe with vented holes that can run an entire length of the plastic bioreactor pipe and serves to provide aeration. The aeration serves to circulate the media for optimal algal growth.

Many modifications and other embodiments of the invention will come to the mind of one skilled in the art having the benefit of the teachings presented in the foregoing descriptions and the associated drawings. Therefore it is to be understood that the invention is not to be limited to the specific embodiments disclosed, and that modifications and alternate embodiments are intended to be included within the scope of the claims supported by this specification.

### EXAMPLE

The following example provides details of certain exemplary methods. As disclosed herein, it is within the scope of the present invention to vary the methods to accommodate variations in location, season, weather, media availability, and bioreactor pipe volume. Accordingly, this example is merely representative of certain embodiments of the invention.

### Example 1

### Growth System Including a 24″×100′ Bioreactor Pipe

Six shallow, parallel troughs oriented from North-to-South are prepared by hand using shovels. The troughs are dug to a depth of approximately 6″ and spaced approximately 8′ apart to minimize shadowing caused by adjacent troughs. The troughs span a substantially flat area with earthen berms on either end. The total length of each of the troughs is approximately 100′. The total area used in the example is approximately 50′ by 100′.

Into each trough is placed an unfilled 100′ bioreactor pipe extruded from polyethylene plastic. The bioreactor pipes are ultraviolet (UV)-resistance treated so as to increase the pipes' resilience to the damaging effects of UV rays.

Within each bioreactor pipe is a tube, such tube containing ports through which pressurized CO₂ is pumped. The tube contains small holes placed through which the pressurized CO₂ passes in order to aerate and agitate the algal culture. Being lighter than the surrounding water, the CO₂/water mix rises, and the culture is both aerated and agitated.

Also within the bioreactor pipe are sensors for measuring pH, temperature, O₂ concentration, CO₂ concentration, NO₃⁻/PO₄³⁻ levels, conductivity, and turbidity. The ends of the bioreactor pipe are placed on the berms such that the ends are above the main length of the bioreactor pipe. The ends of the bioreactor pipe are sealed with cable-ties. Through one end of the bioreactor pipe passes the gas tube as well as cables connecting the pH, temperature, O₂ concentration, CO₂ concentration, NO₃⁻/PO₄³⁻ levels, conductivity, and turbidity sensors within the bioreactor pipe to the control unit outside of the bioreactor pipe. A media inlet port is situated at one end of the bioreactor pipe, and a harvest port is situated at the opposite end of the bioreactor pipe.

Power for the system is provided by a municipal electrical system. The tubular growth system's control unit computer receives its power from the system, as do the various sensors within the bioreactor pipe.

Water for media preparation is provided by a municipal water system. Media is added to the system via a peristaltic pump until the pipe is substantially full, however the ends of the pipe are not completely filled.

CO₂ canisters are attached to the gas line that enters the bioreactor pipe.

The prepared media is added to the bioreactor pipe until the bioreactor pipe is substantially full. After filling the bioreactor pipe, the sensors are “powered up” to ensure they are operating correctly. The bioreactor pipe is inoculated by opening the valve located between the “nursery” bioreactor and the bioreactor pipe. The algal strain used for inoculation is Pseudochlorococcum sp.

The temperature, pH, conductivity, and turbidity of the culture are monitored to ensure that operating parameters are not exceeded. The pH of the culture is maintained at 6.5, and the temperature of the culture is maintained at 25° C.

The culture is allowed to grow until growth slows, as indicated by an increase in turbidity as well as a plateau in cell counts per mL of culture. As algal growth slows, 50% of the culture is harvested through the harvest outlet, and the approximate volume of culture removed from the pipe is replenished through the addition of media through the inlet port.

What is claimed is:

1) A system for growing microorganisms, comprising:
a) a tubular vessel, comprising;
i) a substantially clear flexible, material;
ii) an inlet for adding media;
iii) an outlet for harvesting the microorganisms;
b) an energy source;
c) a media supply; and
d) at least one microorganism selected from the group consisting of Pseudochlorococcum sp., Chlorococcum sp., Chlorella sp., Scenedesmus sp., Palmellococcus sp., Cylindrospermopsis sp., and Planktothrix sp.

2) The system of claim 1 wherein the substantially clear, flexible material comprises polyethylene.

3) The system of claim 1 wherein the substantially clear, flexible material comprises PEEK.

4) The system of claim 1 wherein the substantially clear, flexible material comprises an ultraviolet-resistant material.

5) The system of claim 1 wherein the substantially clear, flexible material is coated to selectively pass specific wavelengths of light.

6) The system of claim 5 wherein the clear, flexible, coated material passes green light and reflects blue light.

7) The system of claim 6 wherein the clear, flexible, coated material passes visible light of a wavelength of about 510 nm and reflects visible light of a wavelength of about 475 nm.

8) The system of claim 5 wherein the clear, flexible, coated material passes blue light and reflects green light.

9) The system of claim 8 wherein the clear, flexible, coated material passes visible light of a wavelength of about 475 nm and reflects visible light of a wavelength of about 510 nm.

10) The system of claim 1 wherein the energy source comprises combustion of the biomass produced by the system.

11) The system of claim 1 wherein the media comprises waste-water.

12) The system of claim 1 wherein the microorganism comprises Chlorella sp.

13) The system of claim 1 wherein the microorganism comprises Pseudochlorococcum sp.

14) A method for growing microorganisms, comprising:
a) adding media to a substantially clear, flexible, tubular vessel with the microorganism;
b) sterilely inoculating the tubular vessel with a microorganism selected from the group consisting of Pseudochlorococcum sp., Chlorococcum sp., Chlorella sp., Scenedesmus sp., Palmellococcus sp., Cylindrospermopsis sp., and Planktothrix sp.;
c) monitoring at least one pre-determined parameter of the culture, selected from the group consisting of: pH, temperature, O₂ concentration, CO₂ concentration, NO₃⁻/PO₄³⁻ levels, conductivity, turbidity; and
d) harvesting at least a part of the culture when the culture exceeds at least one pre-determined parameter selected from the group consisting of: pH, temperature, O₂ concentration, CO₂ concentration, NO₃⁻/PO₄³⁻ levels, conductivity, and turbidity.

15) The system of claim 14 wherein the substantially clear, flexible material comprises polyethylene.

16) The system of claim 14 wherein the substantially clear, flexible material comprises PEEK.

17) The system of claim 14 wherein the substantially clear, flexible material comprises an ultraviolet-resistant material.

18) The system of claim 14 wherein the substantially clear, flexible material is coated to selectively pass specific wavelengths of light.

19) The system of claim 18 wherein the clear, flexible, coated material passes green light and reflects blue light.

20) The system of claim 19 wherein the clear, flexible, coated material passes visible light of a wavelength of about 510 nm and reflects visible light of a wavelength of about 475 nm.

21) The system of claim 18 wherein the clear, flexible, coated material passes blue light and reflects green light.

22) The system of claim 21 wherein the clear, flexible, coated material passes visible light of a wavelength of about 475 nm and reflects visible light of a wavelength of about 510 nm.

23) The system of claim 14 wherein the energy source comprises combustion of the biomass produced by the system.

24) The system of claim 14 wherein the media comprises waste-water.

25) The system of claim 14 wherein the microorganism comprises Chlorella sp.

26) The system of claim 14 wherein the microorganism comprises Pseudochlorococcum sp.

27) An apparatus for growing microorganisms, comprising:
a) a substantially clear, flexible, tubular vessel, wherein said vessel comprises;
i) means for introducing media to the apparatus;
ii) means for introducing at least one microorganism selected from the group consisting of Pseudochlorococcum sp., Chlorococcum sp., Chlorella sp., Scenedesmus sp., Palmellococcus sp., Cylindrospermopsis sp., and Planktothrix sp. to the culture;
iii) means for monitoring at least one parameter of the culture, selected from the group consisting of pH, temperature, O₂ concentration, CO₂ concentration, NO₃⁻/PO₄³⁻ levels, conductivity, and turbidity; and
iv) means for harvesting at least a part of the culture when the culture exceeds at least one pre-determined parameter selected from the group consisting of: pH, temperature, O₂ concentration, CO₂ concentration, NO₃⁻/PO₄³⁻ levels, conductivity, and turbidity.

28) The system of claim 27 wherein the substantially clear, flexible material comprises polyethylene.

29) The system of claim 27 wherein the substantially clear, flexible material comprises PEEK.

30) The system of claim 27 wherein the substantially clear, flexible material comprises an ultraviolet-resistant material.

31) The system of claim 27 wherein the substantially clear, flexible material is coated to selectively pass specific wavelengths of light.

32) The system of claim 31 wherein the clear, flexible, coated material passes green light and reflects blue light.

33) The system of claim 32 wherein the clear, flexible, coated material passes visible light of a wavelength of about 510 nm and reflects visible light of a wavelength of about 475 nm.

34) The system of claim 31 wherein the clear, flexible, coated material passes blue light and reflects green light.

35) The system of claim 34 wherein the clear, flexible, coated material passes visible light of a wavelength of about 475 nm and reflects visible light of a wavelength of about 510 nm.

36) The system of claim 27 wherein the energy source comprises combustion of the biomass produced by the system.

37) The system of claim 27 wherein the media comprises waste-water.

38) The system of claim 27 wherein the microorganism comprises Chlorella sp.

39) The system of claim 27 wherein the microorganism comprises Pseudochlorococcum sp.

40) An apparatus for growing microorganisms, comprising:
a) a plurality of the systems of claim 1, wherein each of the plurality of systems are fluidly-connected to a single bioreactor.
